(19) United States
(12) Patent Application Publication
(10) Pub. No.: US 2008/0166274 A1
(43) Pub. Date: Jul. 10, 2008

(54) Oxidative dehydrogenation of alkyl aromatic hydrocarbons

(75) Inventor: James R. Butler, League City, TX (US)

Correspondence Address: FINA TECHNOLOGY INC, PO BOX 674412, HOUSTON, TX, 77267-4412, US

(73) Assignee: Fina Technology, Inc., Houston, TX (US)

(21) Appl. No.: 11/650,648

(22) Filed: Jan. 8, 2007

(51) Int. Cl. C07C 15/44

(52) U.S. Cl. 422/187; 422/129; 422/188; 585/435

(57) ABSTRACT

Dehydrogenation processes and systems are described herein. In one embodiment, the process generally includes providing an alkyl aromatic hydrocarbon, providing a reaction zone including an oxidative dehydrogenation catalyst, introducing the alkyl aromatic hydrocarbon into the reaction zone, contacting the alkyl aromatic hydrocarbon with the oxidative dehydrogenation catalyst to form a vinyl aromatic hydrocarbon and withdrawing the vinyl aromatic hydrocarbon from the reaction zone. The process generally utilizes a catalyst to oil ratio that is at least 10:1.

### FIELD

Embodiments of the present invention generally relate to dehydrogenation of alkyl aromatic hydrocarbons.

### BACKGROUND

Oxidative dehydrogenation processes generally involve the injection of molecular oxygen into a reaction medium. Although oxidative dehydrogenation may have the same advantages regarding reaction yield and selectivity of the desired product as conventional processes, it is also well known that the presence of molecular oxygen in the reaction medium leads to the formation of undesirable oxidation products, such as aldehydes.

Therefore, a need exists to develop an oxidative dehydrogenation process that does not require the addition of molecular oxygen thereto.

### SUMMARY

Embodiments of the present invention include dehydrogenation processes and systems. In one embodiment, the process generally includes providing an alkyl aromatic hydrocarbon, providing a reaction zone including an oxidative dehydrogenation catalyst, introducing the alkyl aromatic hydrocarbon into the reaction zone, contacting the alkyl aromatic hydrocarbon with the oxidative dehydrogenation catalyst to form a vinyl aromatic hydrocarbon and withdrawing the vinyl aromatic hydrocarbon from the reaction zone. The process generally utilizes a catalyst to oil ratio that is at least 10:1.

In another embodiment, the process generally includes introducing an alkyl aromatic hydrocarbon to a reaction vessel, contacting the alkyl aromatic hydrocarbon with an oxidative dehydrogenation catalyst including a reducible oxide of vanadium supported on a material selected from metallo-silicate zeolites and oxides of a metal selected from Ti, Zr, Zn, Th, Mg, Ca, Ba, Si and Al to form a vinyl aromatic hydrocarbon, wherein the oxidative dehydrogenation catalyst is flowing through the reaction vessel via gravity and withdrawing a vinyl aromatic hydrocarbon and at least a portion of the oxidative dehydrogenation catalyst from the reaction vessel.

The system generally includes a downflow reaction vessel including a first inlet, a second inlet and an outlet, wherein the first inlet is adapted to receive an alkyl aromatic hydrocarbon, the second inlet is adapted to receive an oxidative dehydrogenation catalyst and the outlet is adapted to withdraw a vinyl aromatic hydrocarbon and at least a portion of the oxidative dehydrogenation catalyst therethrough. Further, the oxidative dehydrogenation catalyst includes a reducible oxide of vanadium supported on a material selected from metallo-silicate zeolites and oxides of a metal selected from Ti, Zr, Zn, Th, Mg, Ca, Ba, Si and Al.

### BRIEF DESCRIPTION OF DRAWINGS

FIG. 1 illustrates an embodiment of a dehydrogenation system.

### DETAILED DESCRIPTION

### Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions when the information in this patent is combined with available information and technology.

Various terms as used herein are shown below. To the extent a term used in a claim is not defined below, it should be given the broadest definition persons in the pertinent art have given that term as reflected in printed publications and issued patents. Further, unless otherwise specified, all compounds described herein may be substituted or unsubstituted and the listing of compounds includes derivatives thereof.

As used herein, the term “oxidation state” refers to the degree of oxidation of an atom in a chemical compound. Further, an increase in the oxidation state is generally referred to as oxidation, while a decrease in the oxidation state is generally referred to as reduction.

The term “aldehyde” refers to a compound including an unsaturated carbonyl group.

The term “alkane” refers to an aliphatic hydrocarbon with only single bond.

The term “alkyl” refers to an alkane absent hydrogen.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

Dehydrogenation processes generally include contacting an alkyl aromatic hydrocarbon with a dehydrogenation catalyst to form a vinyl aromatic hydrocarbon. Such contact generally occurs in a reaction zone.

The alkyl aromatic hydrocarbon may include any alkyl aromatic hydrocarbon known to one skilled in the art, such as ethylbenzene, isopropylbenzene or ethyltoluene, for example.

As described herein, the dehydrogenation processes are oxidative dehydrogenation processes. Such processes generally involve the introduction of molecular oxygen into the reaction zone. While oxidative dehydrogenation may have the advantages of high reaction yield and selectivity, it is well known that the presence of molecular oxygen in the reaction zone generally leads to the formation of undesirable oxidation products, such as aldehydes, for example.

Therefore, embodiments of the invention generally utilize a dehydrogenation catalyst in the absence of molecular oxygen. While described herein as a “dehydrogenation catalyst”, it is known to one skilled in the art that the term catalyst as used herein refers to a compound that participates in the dehydrogenation reaction in addition to enhancing the rate of formation of the vinyl aromatic hydrocarbon. Further, the term catalyst may be used interchangeable with the term carrier herein.

The dehydrogenation catalyst may include a reducible oxide of vanadium. As used herein, the term “reducible oxide” refers to an oxide of vanadium which is reduced by contact with hydrocarbons when operating under dehydrogenation conditions.

The dehydrogenation catalyst may optionally be bound to, supported on or extruded with any suitable support material. The support material may include oxides of metals, such as titanium, zirconium, zinc, magnesium, thorium, silica, calcium, barium and aluminum, clays and zeolitic materials, such as metallo-silicates or metallo-alumino-phosphates (e.g., alumino-silicates, borosilicates, silico-alumino-phosphates), for example.

The dehydrogenation catalyst may further include one or more promoters, such as alkali or alkaline-earth metals, for example.

In one specific, non-limiting, embodiment, the dehydrogenation catalyst includes a reducible vanadium oxide on a magnesium oxide support.

The dehydrogenation catalyst may be prepared by methods known to one skilled in the art, such as absorption, precipitation, impregnation or combinations thereof, for example. See, U.S. Pat. No. 5,510,553, which is fully incorporated by reference herein.

The vinyl aromatic hydrocarbon formed via the processes described herein is generally dependent upon the alkyl aromatic hydrocarbon and may include styrene, α-methyl styrene or vinyl toluene, for example. The vinyl aromatic hydrocarbon may further be used for any suitable purpose and/or may undergo further processing, such as separation, for example.

The dehydrogenation processes discussed herein are generally high temperature processes. As used herein, the term “high temperature” refers to process operation temperatures, such as reaction vessel and/or process line temperatures of from about 150° C. to about 1000° C., or from about 300° C. to about 800° C., or from about 500° C. to about 700° C. or from about 550° C. to about 650° C., for example.

Therefore, the alkyl aromatic hydrocarbon may contact the dehydrogenation catalyst in the presence of an inert diluent, such as steam. Such contact may occur in any manner known to one skilled in the art. For example, the diluent may be added to the alkyl aromatic hydrocarbon prior to contact with the catalyst, for example. Although the amount of diluent contacting the alkyl aromatic hydrocarbon is determined by individual process parameters, the diluent may contact the alkyl aromatic hydrocarbon in a weight ratio of from about 0.01:1 to about 15:1, or from about 0.3:1 to about 10:1, or from about 0.6:1 to about 3:1 or from about 1:1 to about 2:1, for example.

In order to maintain selectivity at a desired level, e.g., greater than about 80%, or greater than about 85% or greater than about 90%, it is desirable to maintain the oxidation state of the catalyst within a tolerance of from about 10% to about 20% of the initial oxidation state. As used herein, the term “initial oxidation state” refers to a catalyst particle's oxidation state upon introduction into a reaction zone. Unfortunately, prior oxidative dehydrogenation reactions (e.g., within riser reactors) generally require low catalyst to oil ratios (e.g., 8:1 or less). As used herein, the term catalyst to oil (C:O) refers to the weight ratio of catalyst entering the reaction zone to the alkyl aromatic hydrocarbon (e.g., hydrocarbon/oil) entering the reaction zone. Such catalyst to oil levels generally result in an inability to maintain the oxidation state within desired tolerances. For example, such previous dehydrogenation systems generally experience a reduction rate of from about 20% to about 40%.

However, embodiments of the invention described herein result in the ability to maintain the oxidation state within predetermined tolerances, such as a reduction rate of about 35% or less, or about 30% or less, or about 25% or less, or about 20% or less, or about 15% or less or about 10% or less, for example. Therefore, embodiments of the invention generally utilize a catalyst to oil ratio of at least about 10:1, or from about 15:1 to about 60:1, or from about 20:1 to about 50:1 or from about 25:1 to about 45:1, for example.

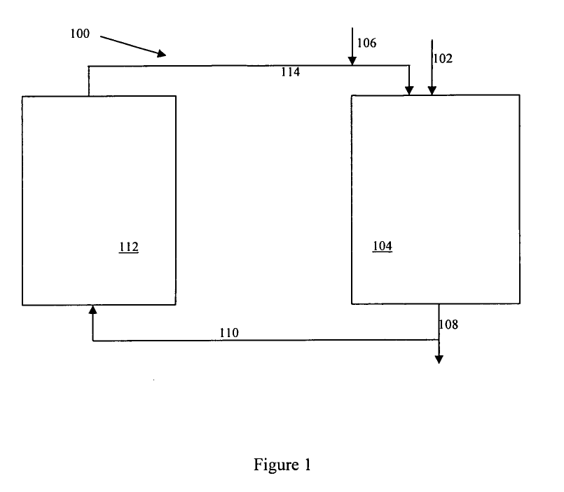

FIG. 1 illustrates a schematic block diagram of a specific, non-limiting, embodiment of a dehydrogenation process 100. Although not shown herein, the process stream flow may be modified based on unit optimization so long as the modification complies with the spirit of the invention, as defined by the claims. For example, additional process equipment, such as heat exchangers or separators, may be employed throughout the processes described herein and such placement is generally known to one skilled in the art. Further, while described below in terms of primary components, the streams indicated below may include any additional components as known to one skilled in the art.

The process 100 generally includes supplying an input stream 102 to a dehydrogenation system 104. The dehydrogenation system 104 is generally adapted to contact the input stream 102 with a dehydrogenation catalyst to form an output stream 108.

The input stream 102 generally includes the alkyl aromatic hydrocarbon and the output stream 108 generally includes the vinyl aromatic hydrocarbon. In addition, the input stream 102 may further include the inert diluent, for example.

The dehydrogenation system 104 generally includes one or more reaction zones, which are contained within one or more reaction vessels. In one embodiment, the reaction vessel generally includes a downflow reaction vessel. As used herein, downflow reaction vessels generally include circulating catalyst therethrough in a downward direction (versus upflow reactors) for contact with a feedstock and recovering the catalyst for regeneration and/or disposal.

Although illustrated as a single reaction zone, it is known to one skilled in the art that the reaction vessel may include one or a plurality or reaction zones, each having catalyst passing therethrough. Further, each reaction zone may be contained within a single reaction vessel or a plurality of reaction vessels, for example.

The dehydrogenation system 104 generally includes feeding the dehydrogenation catalyst to the reaction vessel via line 106 (e.g., fresh catalyst) or line 114 (e.g., regenerated catalyst). The dehydrogenation catalyst is withdrawn from the reaction vessel via an outlet disposed near the bottom of the reaction vessel. The catalyst 106 may be recycled, regenerated and/or disposed of upon withdrawal, for example.

To aid in the flow of the applicable materials, such as steam, input and catalyst, the dehydrogenation system 104 generally utilizes a pressure drop. The applicable materials generally have a short residence time in the reaction zone, further aiding in maintaining the oxidation state in the desired tolerances. For example, the input may have a residence time of from about 0.5 seconds to about 30 seconds or from about 10 seconds to about 15 seconds and the catalyst may have a residence time of from about 0.5 seconds to about 5 minutes or from about 1 second to about 1 minute.

In the embodiment illustrated in FIG. 1, the dehydrogenation catalyst (e.g., separated catalyst) is separated from the vinyl aromatic hydrocarbon and passed via line 110 to a regeneration unit 112. The catalyst is separated from the vinyl aromatic hydrocarbon via suitable means, such as cyclone separation, for example.

The separated catalyst flowing via line 110 is a reduced catalyst. As used herein, the term “reduced catalyst” generally refers to a catalyst that is in a lower oxidation state than the catalyst entering the dehydrogenation unit.

The reduced catalyst may be contacted with a gas stream within a regeneration unit 112 to achieve oxidation of the catalyst and form a regenerated catalyst. The gas stream may include molecular oxygen, for example. In one embodiment, the regeneration unit 112 includes an upflow reaction vessel.

The contact may occur at a temperature of from about 200° C. to about 1000° C. or from about 300° C. to about 900° C., for example. The reduced catalyst may reside in the unit 112 for a time sufficient to at least partially regenerate the catalyst. For example, the reduced catalyst may reside in the regeneration unit 112 for a time of from about 5 seconds to about 5 minutes or from about 10 seconds to about 3 minutes, for example.

As previously discussed, the embodiments described herein generally result in a reduced reduction rate of the dehydrogenation catalyst in comparison to previous oxidative dehydrogenation systems. Therefore, the catalyst generally requires less oxidation for regeneration and greater selectivity control may be obtained.

In one embodiment, at least a portion of the regenerated catalyst is passed via line 114 to the dehydrogenation unit 104.

While illustrated as a downflow reactor (e.g., the flow of input is in the same direction as the flow of the catalyst), the dehydrogenation system may include another flow configuration, such as upflow, for example, so long as the process observes the catalyst to oil ratios described herein.

In one embodiment, the input stream includes a plurality of reactants, which may be fed to the reaction zone via one or more input streams, to form a plurality of products via one or more outlets. The first reactant generally includes the alkyl aromatic hydrocarbon, which forms a vinyl aromatic hydrocarbon product while a second reactant generally includes an alkane, such as ethane, for example. Such a process may include the downflow reactor described herein, or may occur in an alternative reactor capable of utilizing the catalyst and catalyst to oil ratios described herein.

The products may further undergo further processing, such as separation, for example. Such processes generally decrease the production costs of such products, primarily the production costs of ethylene.

While the foregoing is directed to embodiments of the present invention, other and further embodiments of the invention may be devised without departing from the basic scope thereof and the scope thereof is determined by the claims that follow.

What is claimed is:

1. A dehydrogenation process comprising:
providing an alkyl aromatic hydrocarbon;
providing a reaction zone, wherein the reaction zone comprises an oxidative dehydrogenation catalyst;
introducing the alkyl aromatic hydrocarbon into the reaction zone;
contacting the alkyl aromatic hydrocarbon with the oxidative dehydrogenation catalyst to form a vinyl aromatic hydrocarbon; and
withdrawing the vinyl aromatic hydrocarbon from the reaction zone, wherein the process comprises a catalyst to oil ratio that is at least 10:1.

2. The process of claim 1 further comprising withdrawing at least a portion of the oxidative dehydrogenation catalyst from the reaction zone;

3. The process of claim 2 further comprising exposing the portion of the oxidative dehydrogenation catalyst to an oxidation process to form a regenerated catalyst and introducing at least a portion of the regenerated catalyst to the reaction zone.

4. The process of claim 1, wherein the oxidative dehydrogenation catalyst experiences reduction upon contact with the alkyl aromatic hydrocarbon.

5. The process of claim 2, wherein the oxidative dehydrogenation catalyst comprises a first oxidation state upon introduction to the reaction zone and a second oxidation state upon withdrawal from the reaction zone and wherein a difference between the first oxidation state and the second oxidation state is controlled to result in a predetermined selectivity to the vinyl aromatic hydrocarbon.

6. The process of claim 1, wherein the oxidative dehydrogenation catalyst experiences a reduction rate of about 20% or less.

7. The process of claim 1, wherein the alkyl aromatic hydrocarbon comprises ethylbenzene and the vinyl aromatic hydrocarbon comprises styrene.

8. The process of claim 1 further comprising introducing an alkane into the reaction zone.

9. The process of claim 7, wherein the alkyl aromatic hydrocarbon comprises ethylbenzene and the alkane comprises ethane.

10. The process of claim 1, wherein the oxidative dehydrogenation catalyst comprises a reducible oxide of vanadium supported on a material selected from metallo-silicate zeolites and oxides of a metal selected from Ti, Zr, Zn, Th, Mg, Ca, Ba, Si and Al.

11. The process of claim 1, wherein the alkyl aromatic hydrocarbon flows within the reaction zone via gravity.

12. A dehydrogenation system comprising:
a downflow reaction vessel comprising a first inlet, a second inlet and an outlet, wherein the first inlet is adapted to receive an alkyl aromatic hydrocarbon, the second inlet is adapted to receive an oxidative dehydrogenation catalyst and the outlet is adapted to withdraw a vinyl aromatic hydrocarbon and at least a portion of the oxidative dehydrogenation catalyst therethrough; and
wherein the oxidative dehydrogenation catalyst comprises a reducible oxide of vanadium supported on a material selected from metallo-silicate zeolites and oxides of a metal selected from Ti, Zr, Zn, Th, Mg, Ca, Ba, Si and Al.

13. The dehydrogenation system of claim 12, wherein the oxidative dehydrogenation catalyst flows through the reaction vessel via gravity.

14. The dehydrogenation system of claim 12, wherein the alkyl aromatic hydrocarbon flows downflow within the moving bed reaction vessel.

15. The dehydrogenation system of claim 12 further comprising a second downflow reaction vessel.

16. The dehydrogenation system of claim 12 further comprising a regeneration unit adapted to receive the at least a portion of the oxidative dehydrogenation catalyst and at least partially oxidize the catalyst.

17. A dehydrogenation process comprising:
introducing an alkyl aromatic hydrocarbon to a reaction vessel;
contacting the alkyl aromatic hydrocarbon with an oxidative dehydrogenation catalyst comprising a reducible oxide of vanadium supported on a material selected from metallo-silicate zeolites and oxides of a metal selected from Ti, Zr, Zn, Th, Mg, Ca, Ba, Si and Al to form a vinyl aromatic hydrocarbon, wherein the oxidative dehydrogenation catalyst is flowing through the reaction vessel via gravity; and
withdrawing a vinyl aromatic hydrocarbon and at least a portion of the oxidative dehydrogenation catalyst from the reaction vessel.

18. The process of claim 17, wherein the oxidative dehydrogenation catalyst comprises a first oxidation state upon introduction to the reaction vessel and a second oxidation state upon withdrawal from the reaction vessel and wherein a difference between the first oxidation state and the second oxidation state is controlled to result in a predetermined selectivity to the vinyl aromatic hydrocarbon.

19. The process of claim 17, wherein the oxidative dehydrogenation catalyst experiences a reduction rate of about 20% or less.

20. The process of claim 17, wherein the alkyl aromatic hydrocarbon comprises ethylbenzene and the vinyl aromatic hydrocarbon comprises styrene.
